SEQUENCE LISTING (14 sequences)

SEQ ID NO: 1 - protein, 5 aa, Human immunodeficiency virus
TTNYT

SEQ ID NO: 2 - protein, 5 aa, Human immunodeficiency virus
SSTYR

SEQ ID NO: 3 - protein, 5 aa, Human immunodeficiency virus
STNYT

SEQ ID NO: 4 - protein, 5 aa, Human immunodeficiency virus
TTSYT

SEQ ID NO: 5 - protein, 5 aa, Human immunodeficiency virus
NTSYG

SEQ ID NO: 6 - protein, 5 aa, Human immunodeficiency virus
ETWYS

SEQ ID NO: 7 - protein, 5 aa, Human immunodeficiency virus
NTSYR

SEQ ID NO: 8 - protein, 5 aa, Human immunodeficiency virus
INNYT

SEQ ID NO: 9 - protein, 5 aa, Human immunodeficiency virus
IDNYT

SEQ ID NO: 10 - protein, 5 aa, Human immunodeficiency virus
TDNYT

SEQ ID NO: 11 - protein, 5 aa, Human immunodeficiency virus
TDSYS

SEQ ID NO: 12 - protein, 5 aa, Human immunodeficiency virus
TNSTR

SEQ ID NO: 13 - protein, 5 aa, Human immunodeficiency virus
NTRTR

SEQ ID NO: 14 - protein, 8 aa, Human immunodeficiency virus
ASTTTNYT

(19) United States
(12) Patent Application Publication
(10) Pub. No.: US 2018/0344798 A1
(43) Pub. Date: Dec. 6, 2018

(54) TOPICALLY ACTIVE PEPTIDES FOR TREATING COSMETIC AND DERMATOLOGICAL CONDITIONS

(71) Applicant: Creative Bio-Peptides Inc., Potomac, MD (US)

(72) Inventor: Michael R. Ruff, Potomac, MD (US)

(73) Assignee: Creative Bio-Peptides Inc., Potomac, MD (US)

(21) Appl. No.: 15/989,823

(22) Filed: May 25, 2018

Related U.S. Application Data
(60) Provisional application No. 62/512,914, filed on May 31, 2017.

(51) Int. Cl. A61K 38/08; A61K 47/12; A61K 47/22; A61K 47/42; A61K 47/44; A61K 9/06; A61K 9/107; A61K 9/14; A61K 8/64; A61K 8/67; A61K 8/02; A61Q 17/02; A61Q 19/08

(52) U.S. Cl. CPC A61K 38/08; A61K 2800/524; A61K 47/22; A61K 47/42; A61K 47/44; A61K 9/06; A61K 9/107; A61K 9/14; A61K 8/64; A61K 8/671; A61K 8/676; A61K 8/678; A61K 8/022; A61K 8/0229; A61Q 17/02; A61Q 19/08; A61K 2800/436; A61K 2800/48; A61K 47/12

(57) ABSTRACT

A method of treating cosmetic skin conditions in an individual comprising the steps of preparing a composition comprising an all-D amino acid peptide and a pharmaceutically acceptable carrier. The D peptide has the general structure: A-B-C-D-E in which: A is Ser, Thr, Asn, Glu, Arg, Ile, B is Ser, Thr, Asp, Asn, C is Thr, Ser, Asn, Arg, Trp, D is Tyr, and E is Thr, Ser, Arg, or Gly and wherein all amino acids in the D peptide are the D stereoisomeric configuration and said peptide composition is administered in a cosmetically effective dose wherein said composition acts to improve the appearance of the skin. The D peptide may have a terminal amide (—NH₂) or methyl ester moiety to enhance its activity and penetration into the skin.

This application claims benefit to U.S. Provisional Application No. 62/512,914, filed May 31, 2017.

### FIELD OF THE INVENTION

The present invention relates broadly to cosmetic and dermatological formulations containing specific topically active peptides that improve the appearance of skin. The resulting compositions are advantageously used for improving the appearance of skin and reducing redness or irritation which may be caused by burns, cuts, or abrasions, or any other condition, which adversely affect the appearance of aging, wrinkles, redness, dryness, scaly skin, itchiness, and pain sensations.

### BRIEF DESCRIPTION OF THE DRAWINGS

FIGS. 1A-D illustrate that all-D-TTNYT (SEQ ID NO: 1) blocks TLR4-mediated maturation of dendritic cells.

FIG. 2. illustrates that all-D-TTNYT (SEQ ID NO: 1) improved multiple dermatological conditions with topical administration to humans. Self-reported “Severity Scores” improved in dermatocosmetic conditions with topical administration for 28 days in an oil/water lotion.

Table 1 illustrates that DAPTA and the pentapeptide all-D-TTNYT (SEQ ID NO: 1) reduced inflammatory markers in humans, human skin cells, and rodent nerve tissue.

Table 2. illustrates that all-D-TTNYT (SEQ ID NO: 1) reduced neuropathic pain score and shortened the time it took to walk a fixed distance.

In particular embodiments, the invention relates to cosmetic and dermatological conditions with irritation, redness, dryness, sensitivity to pain such as allodynia, whether caused by injury, abrasions, sun exposure, environmental irritants, allergies, microbes, viruses or other infective agents, or of unknown or indeterminate causes.

The number of agents for which topical use has been possible is very small and restricted to lipophilic and low molecular weight substances (nicotinic acid, nitroglycerin, clonidine, steroid hormones, scopolamine). Topical administration of peptides for cosmetic use, which seeks to avoid systemic delivery, but nevertheless must still penetrate into vital layers of skin, has not been broadly successful due to the barrier provided by the skin to penetration of peptides above about 800 d molecular weight. Kraeling, 2014 {Kraeling, 2014} used a small hexa-peptide, of molecular weight approximately 600, and showed that topical administration resulted in only 0.01% penetration into skin epidermis.

An insufficient concentration of a peptide substance applied on the skin may further result from the action of skin proteases which would degrade and inactivate the peptide. As the cellular inflammatory targets reside in the deeper layers of skin, such as the dermis and epidermis, it is essential to be able to deliver a receptor-active peptide into these sites to achieve the desired biological effects. Proteolysis by skin enzymes presents a further challenge to this goal. Protection of cosmetic use peptides to proteolytic degradation in the skin would be desirable for topical utility and for some peptides may be essential.

The significant problems of skin barrier penetration and proteolytic degradation which prevent obtaining effective concentrations are overcome by multiple aspects of the present invention, which is drawn in part to formulations comprising both a protease resistant bioactive peptide, which retains G-protein coupled receptor (GPCR) specificity, reactivity, and potency, with further modifications to enhance delivery into the skin, including small size, less than 600 Kd molecular weight, and in some embodiments introducing a net positive charge of the peptide.

Such combinations enable skin care formulations with three desirable features:

(i) the peptide component provides a specific beneficial bioactivity (e.g., inhibits dendritic cell maturation) to then block the inflammatory transcription factors NfKb and STAT3, with suppression of synthesis of inflammatory cytokines like IL-1, 6, 8 and TNFα and increase anti-inflammatory/regenerative cytokines, like IL-10, with commensurate inhibition of skin redness, itching, swelling etc. leading to improvement in cosmetic appearance.

(ii) the protease resistant feature of the peptides protects the peptide component from degradation, resulting in prolonged beneficial peptide effects, at lower doses, which then,

(iii) coupled with the low molecular weight of the receptor active peptides, allows penetration into the epidermal and dermal skin layers, with may be enhanced with peptide charge cationization, to achieve topically effective doses for beneficial effects on cosmetic skin and dermatological conditions.

Our engineered peptides have multiple desired features that yield in vivo efficacy including potency and receptor-selectivity, penetration into the skin and stability to multiple proteases, all of which then yields a useful invention for topical cosmetic and dermatological use.

Peptides of the present invention represent a pentapeptide minimal bioactive sequence of the octapeptide Peptide T (ASTTTNYT), and its human use analog “DAPTA”, Dalal-peptide T-amide, first described as an antiviral agent by Pert, et al. (U.S. Pat. No. 5,276,016). Further improvements in the present invention have been made to allow penetration into the skin and to confer resistance to enymatic degradation to provide a beneficial cosmetic preparation.

A peptide of the present invention (all-D-TTNYT (SEQ ID NO: 1)) has previously been proposed to be effective in modulating inflammation caused by CCR5 receptors (Appl. Ser. No. 12/688,862, US 2010/0184705 A1). A further use in reducing pain in peripheral neuropathy (Ser. No. 13/024,324), by targeting CCR5, CCR2 and CX3CR1 chemokine receptors, has been disclosed. Neither of these applications teaches a use in topical cosmetic conditions. A prior U.S. Pat. No. 5,248,667 teaches a method of treating psoriasis by use of the peptide “DAPTA” and related D-peptides by oral, buccal, parenteral, topical or rectal administration. The topical administration of DAPTA for skin conditions was not enabled as only intravenous, intranasal and sublingual efficacy was reported. Other published literature using DAPTA in the skin condition psoriasis, such as Farber, 1991 or Marcusson et al., 1989, used intra-lesion injection or intravenous dosing.

Michaelis and Trigg (U.S. Pat. No. 5,798,335) reported prevention or treatment of eczma/dermatitis in dogs with DAPTA. Improvement in two dogs with dermatitis, mild improvement in five cases of erythema, and no improvement in ten cases were described. The effects were marginal. The route of dosing was not described. Other examples of transient effects in dogs used a variety of dosing routes including subcutaneous and dermal spray. DAPTA has a molecular weight of 856 Daltons and would not penetrate skin very well. In this respect Michaelis and Trigg stated a dose of 8.5 mgs/ml is preferable, with doses of 5-1000 mg per day. No cosmetic use in humans is claimed, or anticipated by U.S. Pat. No. 5,798,335. The doses we have employed in treating human cosmetic conditions with the subject peptides are some 100 to 20,000 fold less than preferred for the peptides of Michaelis and Trigg. Those results indicate critical improvements in potency, stability, penetration into skin, or combinations of these features, that are enabled in the present invention.

A further obstacle to the use of DAPTA in a water formulation such as disclosed by Michaelis and Trigg relates to the high propensity for DAPTA to aggregate and lose potency upon storage in aqueous solutions (U.S. Pat. No. 7,390,788), problems not identified in (U.S. Pat. No. 5,798,335). No practical embodiment was therefore disclosed treatment of eczma/dermatitis.

An unexpected and non-obvious aspect of the present invention is the use of all-D amino-acids in the creation of the bioactive peptides.

The result is surprising in view of an earlier study, Pert {Pert, 1986}, FIGS. 3 and 4, which showed that that D for L substitutions in linear peptide ASTTTNYT can cause great loss of potency.

Having one D substitution, in the specific position Nol, (the D-ala) retains potency. The first three N terminal amino acids are not required for activity, and may be modified. The terminal pentapeptide is responsible for the biopotency, and D amino acid modifications of these residues are not favored.

Thus, making an additional D substitution, in the specific position No 8 (the D-Thr) results in loss of 99 to 99.9% of the activity. It is therefore shown that introduction of L to D substitutions can not be made in a general fashion, and that these modifications can, and typically do, destroy biopotency by disrupting the peptide structure required for receptor potency.

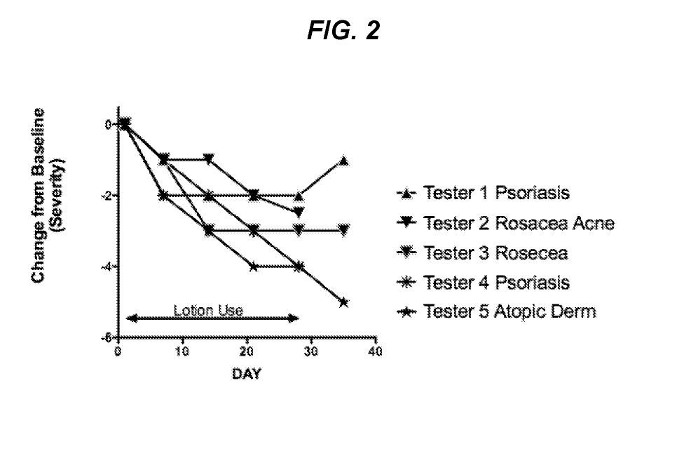

This point is further made in Brenneman, 1988 {Brenneman, 1988, drug}, with specific reference to the peptide TTNYT (SEQ ID NO: 1). See FIG. 2 and Table 1. Upon making the L to D substitution in position 4 (Tyr), the peptide completely loses activity.

A detailed study of the peptide TTNYT (SEQ ID NO: 1) and L to D substitutions was published in Smith, 1988 {Smith et al., 1988, #34265}, Refer to FIG. 3. Introduction of single L to D substitutions in each position 1, 2, 3 and 4 results in loss of potency, and all of the D) form substitutions are substantially less active (50×) to completely inactive.

As such the use of D-substitutions by Andersen in “each” position has not been reduced to practice. The data shows that in no instance does a D for L amino-acid substitution achieve comparable potency to the all-L form, rather D substitutions result in loss of activity, sometimes complete loss of biopotency in a position dependent fashion.

The notion that an all-D peptide would retain significant potency is furthermore novel in consideration of long accepted art of Stewart and Woolley {Stewart and Woolley, 1965, #22293} who prepared all-D peptides. For example, from their article, “In contrast to the change of a single residue, the inversion of all the amino-acid residues in a pentapeptide which has hormonal activity of MSH was found to cause loss of hormonal activity . . . ”

Further in this paper the authors write “because there is as yet no general method for predicting the structural requirements required to make antimetabolites of peptides, we synthesized all-D bradykinin (note 9 amino acids, similar size to the 8 amino acid Formula 1 peptide of Andersen) in an effort to find out whether inversion of all the amino-acids of a peptide may be a generally applicable method for synthesis of peptide antagonsits.”

The authors then concluded: “Amounts of all-D-bradykinin up to 50,000 times the the standard challenge of bradykinin showed neither any inhibition of the response to bradykinin, or any bradykinin-like effect. It would thus seem that inversion of all the amino-acid residues may not be a generally applicable method for formation of antimetabolites of biologically active peptides”.

This conclusion is supported by current art as “loss of function” peptides obtained by inserting D-amino acids is commonly used to define a peptide “pharmacophore”, that is the receptor-binding domain, as a prelude to drug design.

The ability to make D for L amino acid substitutions however creates the possibility to make skin topically deliverable peptide compounds. Stability of peptides in target tissues due to digestive enzymes has limited their utility. The ability to create all-D peptides that retain potency is an unexpected general method of creating peptides of General Formula 1, and likely many others, which may be stabilized to proteolysis, while retaining biopotency, so these peptides benefit from enhanced stability.

Michaelis and Trigg (U.S. Pat. No. 5,798,335) have claimed modified analogs of DAPTA that incorporated D-amino acids. Andersen et al (U.S. Pat. No. 6,011,014 and U.S. Pat. No. 6,265,374) also claim a treatment of inflammation and multiple sclerosis using DAPTA and modified analogs of DAPTA that incorporated D-amino acids. No reduction to practice for any of the D-amino acid modified peptides was provided, and no example of claimed benefit or treatment use with an all-D-amino acid pentapeptide is provided.

Thus neither Pert et al. (US U.S. Pat. No. 5,276,016), who first used D-amino acids in the octapeptide Peptide T (ASTTTNYT) to create the analog DAPTA (Dalal-peptide T-amide), Michaelis and Trigg (U.S. Pat. No. 5,798,335), or Andersen et al (U.S. Pat. No. 6,011,014 and U.S. Pat. No. 6,265,374) teach substitution of all of the naturally occurring L-amino acids by D-amino acids in Peptide T or DAPTA.

The use of D-substitutions in “each” position claimed by Michaelis and Trigg or Andersen et al., cannot be inferred to mean in “all” positions, and in any event has not been reduced to practice. The data of Brennemen, 1998 and Smith, 1988 shows that in no instance does a D for L amino-acid substitution in Sequence ID 1 achieve comparable potency to the all-L form, rather D substitutions result in loss of activity, sometimes complete loss of biopotency in a position dependent fashion.

Thus is cannot be claimed that making all of the amino acids into D-form is obvious. The specific facts relating to the peptides of this invention from the prior published art inform the exact opposite view, that making an all-D peptide would not be efficacious as a cosmetic, or anti-inflammatory agent, or GPCR-active ligand that retains treatment benefits.

Practitioners skilled in the art of peptide design understand that it is overwhelmingly the case that modifications of the peptide backbone, including substitution of D-amino acids, particularly at active sites in the peptide cause loss of activity, and in some modifications complete inactivity. In fact the use of D-amino acid substitutions is commonly used to identify, by loss of function, critical pharmacophore residues in a peptide.

This type of structure-function analysis is a key to drug design and must be determined experimentally in each instance. Our recognition that a pentapeptide fragment of DAPTA (Sequence ID 1) comprised of all-D-amino acids retained substantial biopotency to block receptors was unanticipated in view of Pert, 1986 and Brenneman, 1988 which showed loss of function with D-amino acid substitutions, and complete loss of function with the D-Tyr substitution. We then further determined that other pentapeptides with a Ser, Thr, Asp or Asn in position 2 and a Tyr in position 4 retained activity as all-D-amino acid forms in specific GPCR tests.

The use of all-D-amino acids containing peptides related to SEQ ID NO: 1 that retained substantial biopotency to block CCR5 receptors was first disclosed in U.S. application Ser. No. 12/688,862, however no cosmetic or topical use was enabled.

By the multiple improvements disclosed in this invention a peptide may be topically administered to individuals seeking cosmetic improvement of dermal conditions via skin application and, by virtue of preserved receptor potency, together with small size, together with stability to degradation in skin, together with charge modification to further enhance activity, the subject invention creates an efficacious composition that provides the desired cosmetic benefits.

### Other Active Compounds

Applicant believes other pentapeptides, besides TTNYT (SEQ ID NO: 1), will be effective including the peptides: SSTYR, STNYT, TTSYT, NTSYG, ETWYS, NTSYR, INNYT, IDNYT, TDNYT, TDSYS, TNSYR and NTRYR (SEQ ID NOS: 2-13).

According to a first aspect of the present invention, there is provided the use of a linear peptide wherein all amino acids are in the D-stereoisomeric configuration:

A-B-C-D-E-F-G-H.

wherein:

### A is Ser, Thr, Asn, Glu, Arg, Ile, Leu,

### B is Ser, Thr, Asp, Asn,

### C is Thr, Ser, Asn, Arg, Gln, Lys, Trp,

### D is Tyr, and

### E His Thr, Ser, Arg, Gly.

Candidates for H may be esterified, glycosylated, or amidated.

The modifications of E enhance delivery to target tissues by charge cationization of the peptide at physiological pH in the range of 6 to 8. Previously the terminal amide modification was introduced by Pert et al. to provide protection from carboxypeptidase degradation of DAPTA, and others, including Michaelis and Trigg or Andersen et al., have employed this rationale.

That is not the function here, as Sequence ID 1 is fully protected to degradation and needs no terminal amide (—NH₂), ester, or glycosyl moiety to block proteases. We have determined that a terminal amide (—NH₂) modification in the all-D-peptides of the subject invention enhances its transition across biological membranes or skin barrier and promotes entry into target tissues, such as penetration into the skin. Such modification therefore provides an additional and novel improvement, beyond receptor targeting, small size, and proteolytic stability, to the specific peptides of this invention by enhancing their delivery to target issues.

To test the hypothesis that all-D-peptides which retain receptor activity may be created, with utility in inflammatory conditions, such as may occur in skin or elsewhere in the body, we first used molecular and cellular approaches to explore the inflammatory reaction in isolated immature human monocyte derived immature dendritic cells (iDCs), of which the resting skin Langerhans cell are an example. Our results showed that the maturation markers CD86, HLA-DR, CD58 (adhesion molecule) and ICAM1 (adhesion molecule) are reduced by pre-treatment of the cells with all-D-TTNYT (SEQ ID NO: 1). Similar results were obtained using all-D-TTNYT-NH₂ (SEQ ID NO: 1). The expression of these maturation markers is well known in mediating immune cell trafficking and immune response in the context of injury, skin damage, antigen recognition, host defense, excitotoxicity, and pain nociception, which are relevant to other monocyte derived cells such as the microglia.

We also determined that topical administration in humans with all-D-TTNYT (SEQ ID NO: 1) provided cosmetic and dermatological benefits, and reduced painful sensations in skin. Topical dosing is a preferred cosmetic embodiment which is an improvement over injections or nasal sprays. The modified peptide all-D-TTNYT-NH₂ (SEQ ID NO: 1) had anti-nociceptive effects to subdue painful sensations in the skin of two individuals with diabetes.

The invention will now be illustrated by the following non-limiting examples.

To determine whether all-D-TTNYT (SEQ ID NO: 1) blocks maturation of dendritic cells human PBMC's were isolated from peripheral blood by Ficoll-Paque centrifugation and then monocytes were isolated by negative selection using immunobeads (Miltenyi). Human monocyte derived immature dendritic cells (iDCs) were then generated by treating monocytes with GM-CSF/IL4 for 5 days.

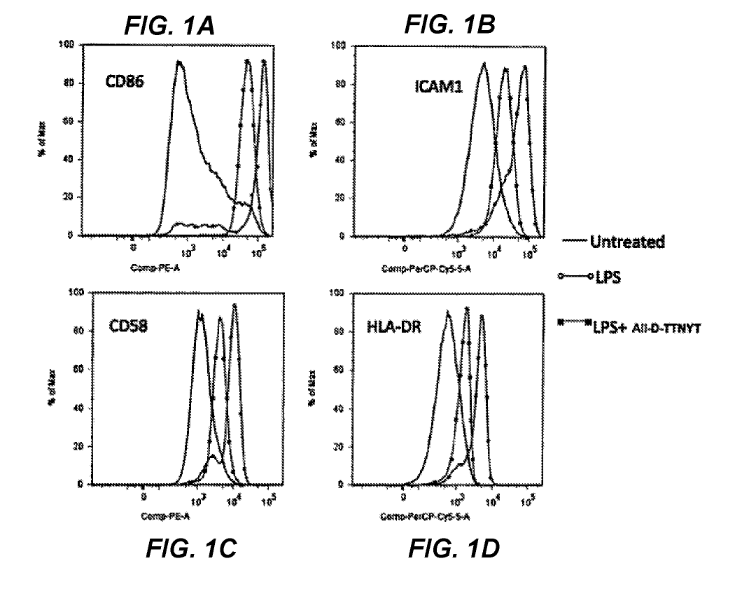

The iDCs were treated with all-D-TTNYT (SEQ ID NO: 1) at 10⁻¹² M for 30 min. After 30 min LPS (100 ng/ml) was added to the cells and cells were analyzed after 48 hrs for surface maturation markers using fluorescent labeled antibodies by flow cytometry. Shown in FIG. 1 are results of CD86, HLA-DR, CD58 (adhesion molecule) and ICAM1 (adhesion molecule) expression induced by TLR4/MyD88 activation (LPS), with and without, added all-D-TTNYT (SEQ ID NO: 1). As seen in the plots pretreatment of cells with all-D-TTNYT (SEQ ID NO: 1) reduces expression of all the maturation markers listed in the figure. The surface markers control T cell activation and localization in tissues. DCs, like microglia, are derived from differentiated monocytes and serve as the innate and adaptive sentinel cells of the skin and brain, respectively. Blockade of DC or microglial activation would suppress inflammation.

All-D-TTNYT (SEQ ID NO: 1) however had no effect on DC maturation of these four markers caused by the antimicrobial peptide LL37, which binds to the insulin-like growth factor 1 receptor (IGF-1R) (not shown). The action of all-D-TTNYT (SEQ ID NO: 1) therefore shows specificity for TLR4/MyD88 induced maturation. TLR4 expression is concentrated to the basal layers in normal skin, whereas it becomes more pronounced in upper layers in diseased skin. The shift in the TLR expression may be related to the disturbed skin barrier and a need for enhanced immune surveillance because of invading microbes, and the results have relevance in atopic dermatitis, contact dermatitis, and psoriasis. We conclude that all-D-TTNYT (SEQ ID NO: 1) and related analogs can have a beneficial effect by modulating dendritic cell activation.

Turning to Table 1 which shows a summary of inflammatory biomarker changes for DAPTA and all-D-TTNYT (SEQ ID NO: 1) relevant to cosmetic improvement of skin aimed toward limiting inflammation. The Table shows results from Ruff, 2013 {Ruff, 2013) which reported reduced plasma inflammatory cytokines (M1-type), and increased anti-inflammatory cytokine IL-10 (M2-type) in subjects who administered DAPTA for 7 weeks; Paoletti, 2013 {Paoletti, 2013} who reported reduced inflammatory markers in normal human epidermal keratinocytes with DAPTA. Padi et al {padi} reported reduced IL-1 and IL-6 in rodent spinal cord tissue with all-D-TTNYT (SEQ ID NO: 1); and reduced activation of human dendritic cells treated with all-D-TTNYT (SEQ ID NO: 1).

To determine whether all-D-TTNYT (SEQ ID NO: 1) has dermatological benefits five subjects self-administered a topical lotion containing an active peptide at 50 μg/ml, 2× per day, with a total daily dose of 45 μms over an area of approximately 10-20 cm² for 28 days.

Self-reported “Severity Scores” on a scale of 1-10 (with 10 being most severe) were recorded in a daily symptom report form to record the symptom score and compliance. The change from baseline is plotted for the day 0, 7, 14, 21, 28 and 35 (one week “off” treatment) symptom scores. Baseline scores ranged from 8 to 4 in the subjects.

Topical administration of all-D-TTNYT (SEQ ID NO: 1) was formulated in an oil/water lotion consisting of deionized water, caprylic/capric triglyceride, polyglyceryl-6-stearate, polyglyceryl-6-behenate, cetearyl alcohol, and glyceryl stearate. Other vehicles, lotions, and creme's known in the cosmetic industry, and even a simple aqueous spray, will be effective carriers for peptide delivery into skin and its deeper layers. Improvements were noted in several dermatocosmetic conditions. The results are shown in FIG. 2. Subjects reported cosmetic normalization of their skin with improvements in the appearance of rosacea, atopic dermatitis, and psoriasis.

To determine whether topical administration of all-D-TTNYT-NH₂ (SEQ ID NO: 1) may benefit a dermatological condition of allodynia two subjects experiencing symptoms consistent with diabetic neuropathy self-administered a topical lotion containing an active peptide at 50 μg/ml, 2× per day, to their feet. Reduced pain sensitivity on the self-reported “Severity Scores” described above was observed. An independent measurement of allodynia benefit, a timed walk over a measured distance was also recorded, and showed improvement after 28 days. (Table 2)

TABLE 1
Summary of Inflammatory Biomarker Changes
for DAPTA and all-D-TTNYT (SEQ ID NO: 1)

| Biomarker | Species | Change | DRUG | Reference |
|---|---|---|---|---|
| IL-1 | Hu | decrease | DAPTA | Ruff, 2003 |
| IL-6 | Hu | decrease | DAPTA | Ruff, 2003 |
| IL-8 | Hu | decrease | DAPTA | Ruff, 2003 |
| TNFα | Hu | decrease | DAPTA | Ruff, 2003 |
| IL-1β | Rat | decrease | all-D-TTNYT | Padi, 2012 |
| | | | (SEQ ID NO: 1) | |
| IL-6 | Rat | decrease | all-D-TTNYT | Padi, 2012 |
| | | | (SEQ ID NO: 1) | |
| IL-6 | Hu | decrease | DAPTA | Paoletti, 2013 |
| IL-8 | Hu | decrease | DAPTA | Paoletti, 2013 |
| IL-23 | Hu | decrease | DAPTA | Paoletti, 2013 |
| ICAM1 | Hu | decrease | DAPTA | Paoletti, 2013 |
| pSTAT3 | Hu | decrease | DAPTA | Paoletti, 2013 |
| CD58 | Hu | decrease | all-D-TTNYT | unpublished |
| | | | (SEQ ID NO: 1) | |
| CD86 | Hu | decrease | all-D-TTNYT | unpublished |
| | | | (SEQ ID NO: 1) | |
| DR | Hu | decrease | all-D-TTNYT | unpublished |
| | | | (SEQ ID NO: 1) | |
| ICAM1 | Hu | decrease | all-D-TTNYT | unpublished |
| | | | (SEQ ID NO: 1) | |

TABLE 2
All-D-TTNYT-NH₂ (SEQ ID NO: 1) reduced neuropathic pain score
and shortened the time it took to walk a fixed distance

| | |
|---|---|
| Pain Score | Timed Walk |

| Subject | Pre | Post | Pre | Post |
|---|---|---|---|---|
| 1 (female, age 57) | 7 | 3 | 48 secs | 24 secs |

| | | | |
|---|---|---|---|
| 2 (female, age 61) | 6 | 3 | Not tested |

What is claimed is:

1: A method of treatment of cosmetic skin conditions in a person comprising the steps of:
preparing a composition comprising a D peptide and a cosmetically acceptable carrier, said D peptide further comprises from five to eight contiguous amino acids having the general structure: A-B-C-D-E-F-G-H in which:
A is Ala, or absent,
B is Ser, Thr or absent,
C is Ser, Thr or absent,
D is Ser, Thr, Asn, Glu, Arg, Ile, Leu,
E is Ser, Thr, Asp, Asn,
F is Thr, Ser, Asn, Arg, Gln, Lys, Trp,
G is Tyr, and
H is Thr, Ser, Arg, Gly,
wherein all amino acids are the D stereoisomeric configuration, and administering said composition to the patient in a cosmetically effective dose, wherein said composition acts to treat skin conditions in the patient.

2: The method as defined in claim 1 wherein said D peptide is TTNYT (SEQ ID NO: 1).

3: The method as defined in claim 1 wherein H may be esterified, glycosylated, or amidated.

4: Preferably the peptide of claim 1 is present in said composition at a concentration between 0.05 to 500 μg/ml.

5: The method as defined in claim 1 wherein said composition can also be formulated in cosmetic vectors such as liposomes, chylomicrons, nanoparticles as well as macro-, micro- and nanocapsules, or be adsorbed on powdered organic polymers, talcs, bentonites and other mineral supports. These solutions or preparations can then be used in creams, lotions, pomades or other cosmetic and dermopharmaceutical preparations.

6: The method as defined in claim 1 wherein said administering said composition to the patient is topical. Preferred topical compositions are skin care compositions, and functional compositions. Topical preparations in accordance with the invention can be in the form of a liquid, lotion, a thickened lotion, a gel, a cream, a milk, an ointment, a paste, a powder, a make-up, or a solid tube stick and can be optionally be packaged as an aerosol and can be provided in the form of a mousse such as an aerosol mousse, a foam or a spray foam, a spray, a stick, a plaster, a cleanser, a soap, a wipe or a lyophilizate. The topical preparations according to the invention are preferably formulated as an oil-in-water or water-in-oil emulsion, water-in-silicone or silicone-in-water emulsion or as an aqueous serum or aqueous gel in particular in as an oil-in water emulsion (O/W emulsion).

7: The method as defined in claim 1 wherein said composition further comprises at least one additional ingredient selected from the group comprising of: vitamin C, derivatives of vitamin C, vitamin E, derivatives of vitamin E, tocopherols, vitamin A, derivatives of vitamin A, retinal, or retinoic acid.

8: The method as defined in claim 1 further comprising, said D peptide is at most eight (8) D amino acid residues in length and contains five contiguous D amino acid residues that have a sequence selected from the group consisting of:
(SEQ ID NO: 1)
Thr Thr Asn Tyr Thr,
(SEQ ID NO: 2)
Ser Ser Thr Tyr Arg,
(SEQ ID NO: 3)
Ser Thr Asn Tyr Thr,
(SEQ ID NO: 4)
Thr Thr Ser Tyr Thr,
(SEQ ID NO: 5)
Asn Thr Ser Tyr Gly,
(SEQ ID NO: 6)
Glu Thr Trp Tyr Ser,
(SEQ ID NO: 7)
Asn Thr Ser Tyr Arg,
(SEQ ID NO: 8)
Ile Asn Asn Tyr Thr,
(SEQ ID NO: 9)
Ile Asp Asn Tyr Thr,
(SEQ ID NO: 10)
Thr Asp Asn Tyr Thr,
(SEQ ID NO: 11)
Thr Asp Ser Tyr Ser,
(SEQ ID NO: 12)
Thr Asn Ser Tyr Arg
and
(SEQ ID NO: 13)
Asn Thr Arg Tyr Arg.

9: The method as defined in claim 1 further comprising an additive selected from the group consisting of: thickeners, fragrances, pearlescent agents, preservatives, sunscreens, anionic or nonionic or cationic or amphoteric polymers, proteins, protein hydrolysates, fatty acids with linear or branched C16-C40 chains, such as 18-methyleicosanoic acid, vitamins, panthenol, volatile or nonvolatile and organomodified or nonorganomodified silicones, vegetable, animal, mineral or synthetic oils and other additives conventionally used in the cosmetics field.

10: The method as defined in claim 1 wherein the said skin condition treatment further comprises improving the esthetic appearance of the skin.
